(19) United States
(12) Patent Application Publication
(10) Pub. No.: US 2014/0100402 A1
(43) Pub. Date: Apr. 10, 2014

(54) Recovery of Olefins from Para-Xylene Process

(71) Applicant: ExxonMobil Chemical Patents Inc., Baytown, TX (US)

(72) Inventors: Allen S. Gawlik, Houston, TX (US); Indulis J. Eilands, League City, TX (US); Terrance C. Osby, Manvel, TX (US)

(21) Appl. No.: 14/035,040

(22) Filed: Sep. 24, 2013

Related U.S. Application Data
(60) Provisional application No. 61/711,346, filed on Oct. 9, 2012.

(51) Int. Cl. C07C 2/86

(52) U.S. Cl. CPC C07C 2/864
USPC 585/467; 585/446

(57) ABSTRACT

A process for producing para-xylene, by (a) contacting toluene with methanol in the presence of an alkylation catalyst under conditions effective to produce an alkylation effluent comprising xylenes and a by-product mixture comprising water, dimethyl ether and C₄− hydrocarbons; (b) separating the alkylation effluent into a first fraction containing xylenes and a second fraction containing the by-product mixture; (c) removing water from the second fraction to produce a dried by-product mixture; (d) fractionating the dried by-product mixture to separate the mixture into a bottoms stream containing dimethyl ether and an overhead stream containing at least some of the C₄- hydrocarbons; and (e) recovering ethylene and propylene from the overhead stream.

### CROSS-REFERENCE TO RELATED APPLICATIONS

This application claims priority to and benefit of U.S. application No. 61/711,346 filed Oct. 9, 2012, the disclosure of which is fully incorporated herein by reference.

### FIELD

This invention relates to a process for producing para-xylene by the alkylation of benzene and/or toluene with methanol and recovery of olefins from the process.

### BACKGROUND

Of the xylene isomers, para-xylene is of particular value since it is useful in the manufacture of terephthalic acid which is an intermediate in the manufacture of synthetic fibers. Para-xylene is a valuable substituted aromatic compound which is in great demand for the production of terephthalic acid, a major component in forming polyester fibers and resins. Today, para-xylene is commercially produced by hydrotreating of naphtha (catalytic reforming), steam cracking of naphtha or gas oil, and toluene disproportionation.

One problem with most existing processes for producing xylenes is that they produce a thermodynamic equilibrium mixture of ortho (o)-, meta (m)- and para (p)-xylenes, in which the para-xylene concentration is typically only about 24 wt %. Thus, separation of para-xylene from such mixtures tends to require superfractionation and multistage refrigeration steps. Such processes involve high operation costs and result in only limited yields. There is therefore a continuing need to provide processes which are highly selective for the production of p-xylene.

One known method for producing xylenes involves the alkylation of benzene and/or toluene with methanol over a solid acid catalyst. Thus the alkylation of toluene with methanol over cation-exchanged zeolite Y has been described by Yashima et al. in the Journal of Catalysis 16, 273-280 (1970). These workers reported selective production of para-xylene over the approximate temperature range of 200 to 275° C., with the maximum yield of para-xylene in the mixture of xylenes, i.e., about 50% of the xylene product mixture, being observed at 225° C. Higher temperatures were reported to result in an increase in the yield of meta-xylene and a decrease in production of para and ortho-xylenes.

Alternatively, ZSM-5-type zeolite, zeolite beta and silicaaluminophosphate (SAPO) catalysts have been used for this process. For example, U.S. Pat. No. 6,504,072 discloses a toluene methylation process that is highly selective for the production of para-xylene and which comprises reacting toluene with methanol in the presence of a catalyst comprising a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa). The porous crystalline material is preferably a medium-pore zeolite, particularly ZSM-5, which has been severely steamed at a temperature of at least 950° C. The porous crystalline material is preferably combined with at least one oxide modifier, preferably including phosphorus, to control reduction of the micropore volume of the material during the steaming step.

Although toluene methylation, and particularly the para-selective toluene methylation process of U.S. Pat. No. 6,504,072, provides an attractive route to para-xylene, the process inevitably produces significant quantities of light (C₄−) gas. These gaseous by-products include olefins, particularly ethylene, propylene and butylenes; alkanes, such as methane, ethane, propane and butanes; and oxygenates, such as dimethyl ether. To improve overall process economics, there is a need for an efficient method of recovering at least some of these by-products so that the value of the light gas stream can be increased above fuel value. The present invention seeks to provide such a process.

According to the present invention, it has now been found that significant quantities of light olefins, particularly ethylene and propylene, can be recovered from methanol/toluene alkylation processes and diverted to uses other than merely as fuel.

### SUMMARY

The invention resides in a process for producing para-xylene, the process comprising (a) contacting benzene and/or toluene with methanol in the presence of an alkylation catalyst under conditions effective to produce an alkylation effluent comprising xylenes and a by-product mixture comprising water, dimethyl ether and C₄− hydrocarbons; (b) separating the alkylation effluent into a first fraction containing xylenes and a second fraction containing the by-product mixture; (c) removing water from the second fraction to produce a dried by-product mixture; (d) fractionating the dried by-product mixture to separate the mixture into a bottoms stream containing dimethyl ether and an overhead stream containing at least some of the C₄− hydrocarbons; and (e) recovering ethylene and propylene from the overhead stream.

Advantageously, the process can be conducted such that water is removed from said second fraction by passing the second fraction through a molecular sieve drier, or in the alternative, such that water is removed from said second fraction by washing the second fraction with methanol.

In a preferred embodiment, the process includes passing the methanol through a molecular sieve drier prior to washing the second fraction with the methanol and can be conducted such that the dried by-product mixture comprises less than 100 ppm by weight of water, more preferably 20 ppm by weight, or less, of water, and most preferably 1 ppm by weight, or less, of water, and the overhead stream comprises less than 100 ppm by weight of dimethyl ether, more preferably 20 ppm by weight, or less, of dimethyl ether, and most preferably 1 ppm by weight, or less, of dimethyl ether.

In another embodiment, the by-product mixture produced in step (a) also comprises carbon monoxide, and the process further comprises removing the carbon monoxide from the by-product mixture prior to the water removal step (c), or removing carbon monoxide from the overhead stream, which can be in the vapor phase, prior to the recovery step (e).

In another embodiment, the process is conducted such that the overhead stream is a vapor-phase stream, and can further comprise (d)(i) passing the overhead stream of step (d) in a vapor phase into a partial condenser and cooling the stream to remove remaining condensables from the vapor phase; and (d)(ii) recovering the vapor phase, and advantageously (d)(iii) passing the vapor phase from step (d)(ii) to a cryogenic separation unit to separate ethylene and propylene from any remaining overhead stream components. In an embodiment, ethylene and propylene can be recovered separately or together, such as cryogenically, to be used, for instance in further processing steps(e.g., polymerization) or for sale.

Conveniently, the cryogenic separation unit is one of a refinery gas recovery unit or a fluidized catalytic cracking unit recovery unit.

In a particularly preferred embodiment, step (a) of the process includes providing a feedstream containing at least about 90 wt % toluene, and can be conducted over an alkylation catalyst which is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa), such as a medium-pore size aluminosilicate zeolite selected from the group consisting of ZSM-5, ZSM-11, ZSM-12, ZSM-22, ZSM-23, ZSM-35, and ZSM-48, optionally composited with an inorganic oxide matrix.

In another embodiment, the process can be conducted such that a methanol feed is injected in stages into the alkylation catalyst at one or more locations downstream from the location of injection of the toluene.

### BRIEF DESCRIPTION OF THE DRAWINGS

FIG. 1 is a flow diagram of a process for recovery of light olefins from a methanol/toluene alkylation process, according to one example of the present application.

### DETAILED DESCRIPTION OF THE EMBODIMENTS

Described herein is a process for producing para-xylene by the catalytic alkylation of benzene and/or toluene with methanol. The alkylation process produces a para-rich mixture of xylene isomers, together with water and some light organic by-products, particularly dimethyl ether and C₄− olefinic hydrocarbons. The present process provides an improved method of separating and recovering at least the olefins from these light by-products for uses other than as fuel.

### Alkylation Process

The alkylation process employed herein can employ any aromatic feedstock comprising benzene and/or toluene, although in general it is preferred that the aromatic feed contains at least 90 wt %, especially at least 99 wt %, of toluene. Similarly, although the composition of the methanol-containing feed is not critical, it is generally desirable to employ feeds containing at least 90 wt %, especially at least 99 wt %, of methanol.

The catalyst employed in the alkylation process is generally a porous crystalline material and, in one preferred embodiment, is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa).

As used herein, the Diffusion Parameter of a particular porous crystalline material is defined as D/r²×10⁶, wherein D is the diffusion coefficient (cm²/sec) and r is the crystal radius (cm). The diffusion parameter can be derived from sorption measurements provided the assumption is made that the plane sheet model describes the diffusion process. Thus for a given sorbate loading Q, the value Q/Q_{eq}, where Q_{eq} is the equilibrium sorbate loading, is mathematically related to (Dt/r²)^{1/2} where t is the time (sec) required to reach the sorbate loading Q. Graphical solutions for the plane sheet model are given by J. Crank in “The Mathematics of Diffusion”, Oxford University Press, Ely House, London, 1967.

The porous crystalline material is preferably a medium-pore size aluminosilicate zeolite. Medium pore zeolites are generally defined as those having a pore size of about 5 to about 7 Angstroms, such that the zeolite freely sorbs molecules such as n-hexane, 3-methylpentane, benzene and p-xylene. Another common definition for medium pore zeolites involves the Constraint Index test which is described in U.S. Pat. No. 4,016,218, which is incorporated herein by reference. In this case, medium pore zeolites have a Constraint Index of about 1-12, as measured on the zeolite alone without the introduction of oxide modifiers and prior to any steaming to adjust the diffusivity of the catalyst. In addition to the medium-pore size aluminosilicate zeolites, other medium pore acidic metallosilicates, such as silicoaluminophosphates (SAPOs), can be used in the present process.

Particular examples of suitable medium pore zeolites include ZSM-5, ZSM-11, ZSM-12, ZSM-22, ZSM-23, ZSM-35, and ZSM-48, with ZSM-5 and ZSM-11 being particularly preferred. In one embodiment, the zeolite employed in the process of the invention is ZSM-5 having a silica to alumina molar ratio of at least 250, as measured prior to any treatment of the zeolite to adjust its diffusivity.

Zeolite ZSM-5 and the conventional preparation thereof are described in U.S. Pat. No. 3,702,886. Zeolite ZSM-11 and the conventional preparation thereof are described in U.S. Pat. No. 3,709,979. Zeolite ZSM-12 and the conventional preparation thereof are described in U.S. Pat. No. 3,832,449. Zeolite ZSM-23 and the conventional preparation thereof are described U.S. Pat. No. 4,076,842. Zeolite ZSM-35 and the conventional preparation thereof are described in U.S. Pat. No. 4,016,245. ZSM-48 and the conventional preparation thereof is taught by U.S. Pat. No. 4,375,573. The entire disclosures of these U.S. patents are incorporated herein by reference.

The medium pore zeolites described above are preferred for the present process since the size and shape of their pores favor the production of p-xylene over the other xylene isomers. However, conventional forms of these zeolites have Diffusion Parameter values in excess of the 0.1-15 sec⁻¹ range desired for the present process. Nevertheless, the required diffusivity can be achieved by severely steaming the zeolite so as to effect a controlled reduction in the micropore volume of the catalyst to not less than 50%, and preferably 50-90%, of that of the unsteamed catalyst. Reduction in micropore volume is monitored by measuring the n-hexane adsorption capacity of the zeolite, before and after steaming, at 90° C. and 75 torr n-hexane pressure.

Steaming to achieve the desired reduction in the micropore volume of the porous crystalline material can be effected by heating the material in the presence of steam at a temperature of at least about 950° C., preferably about 950 to about 1075° C., and most preferably about 1000 to about 1050° C. for about 10 minutes to about 10 hours, preferably from 30 minutes to 5 hours.

To effect the desired controlled reduction in diffusivity and micropore volume, it may be desirable to combine the porous crystalline material, prior to steaming, with at least one oxide modifier, preferably selected from oxides of the elements of Groups IIA, IIIA, IIIB, IVA, VA, VB and VIA of the Periodic Table (IUPAC version). Conveniently, said at least one oxide modifier is selected from oxides of boron, magnesium, calcium, lanthanum and preferably phosphorus. In some cases, it may be desirable to combine the porous crystalline material with more than one oxide modifier, for example a combination of phosphorus with calcium and/or magnesium, since in this way it may be possible to reduce the steaming severity needed to achieve a target diffusivity value. The total amount of oxide modifier present in the catalyst, as measured on an elemental basis, may be between about 0.05 and about 20 wt %, such as between about 0.1 and about 10 wt %, based on the weight of the final catalyst.

Where the modifier includes phosphorus, incorporation of modifier in the alkylation catalyst is conveniently achieved by the methods described in U.S. Pat. Nos. 4,356,338; 5,110,776; 5,231,064 and 5,348,643, the entire disclosures of which are incorporated herein by reference. Treatment with phosphorus-containing compounds can readily be accomplished by contacting the porous crystalline material, either alone or in combination with a binder or matrix material, with a solution of an appropriate phosphorus compound, followed by drying and calcining to convert the phosphorus to its oxide form. Contact with the phosphorus-containing compound is generally conducted at a temperature of about 25° C. and about 125° C. for a time between about 15 minutes and about 20 hours. The concentration of the phosphorus in the contact mixture may be between about 0.01 and about 30 wt %.

Representative phosphorus-containing compounds which may be used to incorporate a phosphorus oxide modifier into the catalyst of the invention include derivatives of groups represented by PX₃, RPX₂, R₂PX, R₃P, X₃PO, (XO)₃PO, (XO)₃P, R₃P═O, R₃P═S, RPO₂, RPS₂, RP(O)(OX)₂, RP(S)(SX)₂, R₂P(O)OX, R₂P(S)SX, RP(OX)₂, RP(SX)₂, ROP(OX)₂, RSP(SX)₂, (RS)₂PSP(SR)₂, and (RO)₂POP(OR)₂, where R is an alkyl or aryl, such as phenyl radical, and X is hydrogen, R, or halide. These compounds include primary, RPH₂, secondary, R₂PH, and tertiary, R₃P, phosphines such as butyl phosphine, the tertiary phosphine oxides, R₃PO, such as tributyl phosphine oxide, the tertiary phosphine sulfides, R₃PS, the primary, RP(O)(OX)₂, and secondary, R₂P(O)OX, phosphonic acids such as benzene phosphonic acid, the corresponding sulfur derivatives such as RP(S)(SX)₂ and R₂P(S)SX, the esters of the phosphonic acids such as dialkyl phosphonate, (RO)₂P(O)H, dialkyl alkyl phosphonates, (RO)₂P(O)R, and alkyl dialkylphosphinates, (RO)P(O)R₂; phosphinous acids, R₂POX, such as diethylphosphinous acid, primary, (RO)P(OX)₂, secondary, (RO)₂POX, and tertiary, (RO)₃P, phosphites, and esters thereof such as the monopropyl ester, alkyl dialkylphosphinites, (RO)PR₂, and dialkyl alkyphosphinite, (RO)₂PR, esters. Corresponding sulfur derivatives may also be employed including (RS)₂P(S)H, (RS)₂P(S)R, (RS)P(S)R₂, R₂PSX, (RS)P(SX)₂, (RS)₂PSX, (RS)₃P, (RS)PR₂, and (RS)₂PR. Examples of phosphite esters include trimethylphosphite, triethylphosphite, diisopropylphosphite, butylphosphite, and pyrophosphites such as tetraethylpyrophosphite. The alkyl groups in the mentioned compounds preferably contain one to four carbon atoms.

Other suitable phosphorus-containing compounds include ammonium hydrogen phosphate, the phosphorus halides such as phosphorus trichloride, bromide, and iodide, alkyl phosphorodichloridites, (RO)PCl₂, dialkylphosphoro-chloridites, (RO)₂PCl, dialkylphosphinochloroidites, R₂PCl, alkyl alkylphosphonochloridates, (RO)(R)P(O)Cl, dialkyl phosphinochloridates, R₂P(O)Cl, and RP(O)Cl₂. Applicable corresponding sulfur derivatives include (RS)PCl₂, (RS)₂PCl, (RS)(R)P(S)Cl, and R₂P(S)Cl.

Particular phosphorus-containing compounds include ammonium phosphate, ammonium dihydrogen phosphate, diammonium hydrogen phosphate, diphenyl phosphine chloride, trimethylphosphite, phosphorus trichloride, phosphoric acid, phenyl phosphine oxychloride, trimethylphosphate, diphenyl phosphinous acid, diphenyl phosphinic acid, diethylchlorothiophosphate, methyl acid phosphate, and other alcohol-P₂O₅ reaction products.

Representative boron-containing compounds which may be used to incorporate a boron oxide modifier into the catalyst of the invention include boric acid, trimethylborate, boron oxide, boron sulfide, boron hydride, butylboron dimethoxide, butylboric acid, dimethylboric anhydride, hexamethylborazine, phenyl boric acid, triethylborane, diborane and triphenyl boron.

Representative magnesium-containing compounds include magnesium acetate, magnesium nitrate, magnesium benzoate, magnesium propionate, magnesium 2-ethylhexoate, magnesium carbonate, magnesium formate, magnesium oxylate, magnesium bromide, magnesium hydride, magnesium lactate, magnesium laurate, magnesium oleate, magnesium palmitate, magnesium salicylate, magnesium stearate and magnesium sulfide.

Representative calcium-containing compounds include calcium acetate, calcium acetylacetonate, calcium carbonate, calcium chloride, calcium methoxide, calcium naphthenate, calcium nitrate, calcium phosphate, calcium stearate and calcium sulfate.

Representative lanthanum-containing compounds include lanthanum acetate, lanthanum acetylacetonate, lanthanum carbonate, lanthanum chloride, lanthanum hydroxide, lanthanum nitrate, lanthanum phosphate and lanthanum sulfate.

The porous crystalline material employed in the process of the invention may be combined with a variety of binder or matrix materials resistant to the temperatures and other conditions employed in the process. Such materials include active and inactive materials such as clays, silica and/or metal oxides such as alumina. The latter may be either naturally occurring or in the form of gelatinous precipitates or gels including mixtures of silica and metal oxides. Use of a material which is active, tends to change the conversion and/or selectivity of the catalyst and hence is generally not preferred. Inactive materials suitably serve as diluents to control the amount of conversion in a given process so that products can be obtained economically and orderly without employing other means for controlling the rate of reaction. These materials may be incorporated into naturally occurring clays, e.g., bentonite and kaolin, to improve the crush strength of the catalyst under commercial operating conditions. Said materials, i.e., clays, oxides, etc., function as binders for the catalyst. It is desirable to provide a catalyst having good crush strength because in commercial use it is desirable to prevent the catalyst from breaking down into powder-like materials. These clay and/or oxide binders have been employed normally only for the purpose of improving the crush strength of the catalyst.

Naturally occurring clays which can be composited with the porous crystalline material include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or initially subjected to calcination, acid treatment or chemical modification.

In addition to the foregoing materials, the porous crystalline material can be composited with a porous matrix material such as silica-alumina, silica-magnesia, silica-zirconia, silica-thoria, silica-beryllia, silica-titania as well as ternary compositions such as silica-alumina-thoria, silica-alumina-zirconia silica-alumina-magnesia and silica-magnesia-zirconia.

The relative proportions of porous crystalline material and inorganic oxide matrix vary widely, with the content of the former ranging from about 1 to about 90% by weight and more usually, particularly when the composite is prepared in the form of beads, in the range of about 2 to about 80 wt % of the composite.

The alkylation process can be conducted in any known reaction vessel but generally the methanol and aromatic feeds are contacted with the catalyst described above with the catalyst particles being disposed in one or more fluidized beds. Each of the methanol and aromatic feeds can be injected into the fluidized catalyst in a single stage. However, in one embodiment, the methanol feed is injected in stages into the fluidized catalyst at one or more locations downstream from the location of the injection of the aromatic reactant into the fluidized catalyst. For example, the aromatic feed can be injected into a lower portion of a single vertical fluidized bed of catalyst, with the methanol being injected into the bed at a plurality of vertically spaced intermediate portions of the bed and the product being removed from the top of the bed. Alternatively, the catalyst can be disposed in a plurality of vertically spaced catalyst beds, with the aromatic feed being injected into a lower portion of the first fluidized bed and part of the methanol being injected into an intermediate portion of the first bed and part of the methanol being injected into or between adjacent downstream catalyst beds.

The conditions employed in the alkylation stage of the present process are not narrowly constrained but, in the case of the methylation of toluene, generally include the following ranges: (a) temperature between about 500 and about 700° C., such as between about 500 and about 600° C.; (b) pressure of between about 1 atmosphere and about 1000 psig (between about 100 and about 7000 kPa), such as between about 10 psig and about 200 psig (between about 170 and about 1480 kPa); (c) moles toluene/moles methanol (in the reactor charge) of at least about 0.2, such as from about 0.2 to about 20; and (d) a weight hourly space velocity (“WHSV”) for total hydrocarbon feed to the reactor(s) of about 0.2 to about 1000, such as about 0.5 to about 500 for the aromatic reactant, and about 0.01 to about 100 for the combined methanol reagent stage flows, based on total catalyst in the reactor(s).

### Product Treatment and Recovery

The product of the reaction between the methanol and toluene and/or benzene is an alkylation effluent comprising para-xylene and other xylene isomers, water vapor, unreacted toluene and/or benzene, unreacted methanol, phenolic impurities, and a variety of light gas by-products, such as C₄− hydrocarbons, including light olefins, and dimethyl ether. The alkylation effluent will also generally contain some C₉+ aromatic by-products. In addition, where the process is conducted in a fluidized catalyst bed, the alkylation effluent will contain some entrained solid catalyst and catalyst fines. Thus the effluent, which is generally in the vapor phase, leaving the (final) fluidized bed reactor is generally passed through an integral cyclone separator to remove some of the entrained catalyst solids and return them to the alkylation reactor.

The alkylation effluent leaves the alkylation reactor system at a high temperature, typically between about 500 and about 600° C. and initially may be passed through a heat exchanger so that the waste heat in the effluent stream may be recovered and used to heat other process stream(s). It is, however, preferred that any initial cooling of the product stream is limited so as to keep the effluent vapors well above the dew point, typically about 240° F. (116° C.).

Following further cooling, the effluent vapor stream is fed to a separation system, which may comprise one or more fractionation columns, where the unreacted methanol and aromatics are recovered and recycled to the alkylation step, the light (C₄−) and heavy (C₉+) by-products are removed and the remainder of effluent is separated into a liquid organic product stream rich in xylenes and a waste water stream. The waste water is decanted from the organic product stream and the para-xylene is recovered from the organic product stream, typically by fractional crystallization or selective adsorption.

In the present process, rather than being sent directly to fuel use, the light (C₄−) stream is treated to recover at least the valuable olefinic component of the stream. Typically, this treatment initially involves subjecting the light stream to a drying step to remove water, such as with a molecular sieve drier or by washing with methanol, which itself has preferably been dried to remove water, such as with a molecular sieve drier. The dried by-product mixture preferably contains less than 100 ppm water by weight, preferably 20 ppm or less by weight, still more preferably 1 ppm or less by weight. Optionally, some or all of the carbon monoxide formed in the methylation process can be removed prior to the drying step. The amount of water present can be conveniently measured by a Panametrics moisture analyzer, gas chromatograph, simulation using VLE data, and other methods known by those of ordinary skill in the art.

The dried by-product mixture is then sent to a fractionation tower primarily to remove dimethyl ether from the light olefins, so as to minimize the impact of dimethyl ether on olefins recovery equipment. Dimethyl ether can also be deleterious to a later-recovered propylene product by negatively impacting propylene in downstream processes such as polymerization. The fractionation tower acts to fractionate the dried by-product mixture into an overhead stream, containing at least some of the C₄− hydrocarbons, and almost all of the dimethyl ether and C₄+ hydrocarbons as a liquid bottoms stream. For example, ethylene and at least about 98 wt % of the propylene, and about 67 wt % of the propane from the fractionation column are recovered in the overhead stream, while nearly 100 wt % of the dimethyl ether and nearly 100 wt % of C₄+ hydrocarbons are removed in the liquid bottoms stream. The overhead vapor from the fractionation tower, which generally comprises less than about 100 ppm dimethyl ether, preferably 20 ppm or less by weight, more preferably 1 ppm or less by weight, and if not previously removed some carbon monoxide, is sent to a refrigerated partial condenser, such as a condenser using propylene refrigeration, and then to a separation drum, where a vapor product phase is recovered and remaining condensables are removed and provided as reflux to the fractionation tower. This arrangement avoids the need for the ethylene refrigeration which would be required to condense all the vapor from the tower overhead and thereby decreases cooling costs. The amount of DME in the overhead vapor is most preferably analyzed by gas chromatography which has been calibrated to measure oxygenates such as DME.

Subsequently, the vapor product phase recovered from the separation drum is passed to a cryogenic separation unit, preferably an existing cryogenic separation unit associated with, for example, a refinery gas recovery system or a fluidized catalytic cracking unit recovery system, to effect separation and recovery of ethylene and propylene from any remaining gases in the overhead vapor. If not previously removed, carbon monoxide is removed prior to sending the overhead vapor phase to the cryogenic separation unit.

The dimethyl ether and C₄+ hydrocarbons removed from the fractionation tower as the liquid bottoms stream can be used as fuel or the dimethyl ether can be recovered using a further fractionation tower and recycled to the methylation reactor.

The carbon monoxide recovered from the methylation reaction effluent can also be used as fuel or can be converted to methane in a conventional methanation reactor according to the following reaction:

CO+3H₂→CH₄+H₂O

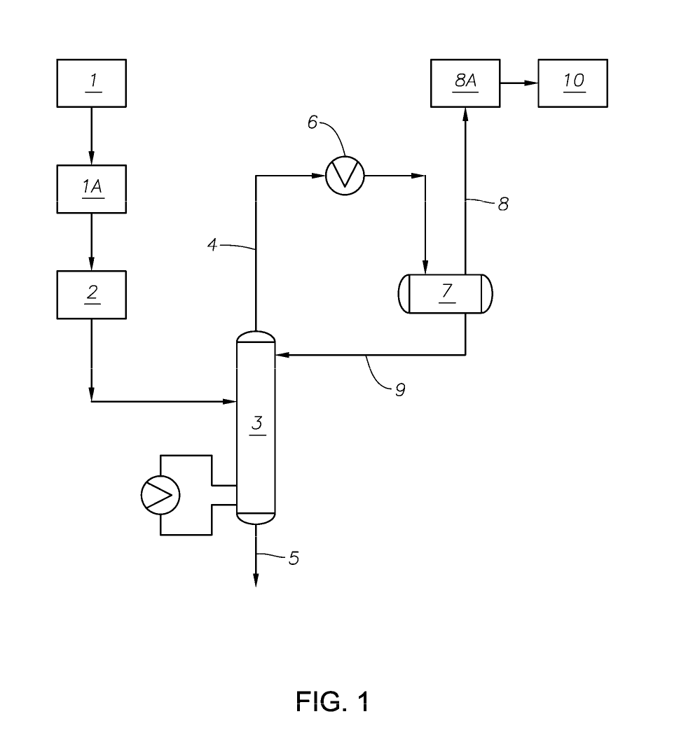

One example of the present process for treating the light gases from a toluene methylation unit is shown in FIG. 1, in which the light gases (1) are fed via an optional CO recovery unit (1A) to a dryer (2). After passage through the dryer (2), the dried gaseous mixture is sent to a fractionation tower (3), where the mixture is divided into an overhead vapor stream (4) and a liquid bottoms stream (5). The overhead vapor stream (4) is sent to a refrigerated partial condenser (6), such as a condenser using propylene refrigeration, and then to a separation drum (7), wherein a vapor phase (8) is recovered and the remaining condensables are removed and provided as reflux (9) to the fractionation tower. The vapor phase (8) is then fed via an optional CO recovery unit (8A) to a cryogenic separation unit (10) for recovery of the ethylene and propylene.

While the present invention has been described and illustrated by reference to particular embodiments, those of ordinary skill in the art will appreciate that the invention lends itself to variations and modifications not necessarily illustrated herein without departing from the spirit and scope of the invention.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated.

1. A process for producing para-xylene, the process comprising:
(a) contacting toluene and/or benzene with methanol in the presence of an alkylation catalyst under conditions effective to produce an alkylation effluent comprising xylenes and a by-product mixture comprising water, dimethyl ether and C₄− hydrocarbons;
(b) separating the alkylation effluent into a first fraction containing xylenes and a second fraction containing the by-product mixture;
(c) removing water from the second fraction to produce a dried by-product mixture;
(d) fractionating the dried by-product mixture to separate the mixture into a bottoms stream containing dimethyl ether and an overhead stream containing at least some of the C₄− hydrocarbons; and
(e) recovering ethylene and propylene from the overhead stream.

2. The process of claim 1, wherein water is removed from said second fraction by passing the second fraction through a molecular sieve drier.

3. The process of claim 1, wherein water is removed from said second fraction by washing the second fraction with methanol.

4. The process of claim 3, further comprising passing the methanol through a molecular sieve drier prior to washing the second fraction with the methanol.

5. The process of claim 1, wherein the dried by-product mixture comprises less than 100 ppm by weight of water.

6. The process of claim 1, wherein the overhead stream comprises less than 100 ppm by weight of dimethyl ether.

7. The process of claim 1, wherein the by-product mixture produced in (a) also comprises carbon monoxide.

8. The process of claim 7, further comprising removing carbon monoxide from the by-product mixture prior to the water removal step (c).

9. The process of claim 7, further comprising removing carbon monoxide from the overhead stream prior to the recovery step (e).

10. The process of claim 1, wherein the overhead stream is a vapor-phase stream.

11. The process of claim 1, further comprising
(d)(i) passing the overhead stream of step (d) in a vapor phase into a partial condenser and cooling the stream to remove remaining condensables from the vapor phase; and
(d)(ii) recovering the vapor phase.

12. The process of claim 11, further comprising
(d)(iii) passing the vapor phase from step (d)(ii) to a cryogenic separation unit to separate ethylene and propylene from any remaining overhead stream components and optionally further including a step (d) (iv) of separating ethylene from propylene cryogenically.

13. The process of claim 12, wherein the cryogenic separation unit is one of a refinery gas recovery system or a fluidized catalytic cracking unit recovery system, or an ethylene plant recovery system, or a pyrolysis cracking furnace system, or any combination thereof.

14. The process of claim 12, further comprising removing carbon monoxide from the vapor phase.

15. The process of claim 13, further comprising removing carbon monoxide from the vapor phase.

16. The process of claim 1, wherein the toluene is provided in a feedstream containing at least about 90 wt % toluene.

17. The process of claim 1, wherein the alkylation catalyst is a porous crystalline material having a Diffusion Parameter for 2,2 dimethylbutane of about 0.1-15 sec⁻¹ when measured at a temperature of 120° C. and a 2,2 dimethylbutane pressure of 60 torr (8 kPa).

18. The process of claim 17, wherein the alkylation catalyst is a medium-pore size aluminosilicate zeolite selected from the group consisting of ZSM-5, ZSM-11, ZSM-12, ZSM-22, ZSM-23, ZSM-35, and ZSM-48, optionally composited with an inorganic oxide matrix.

19. The process of claim 1, wherein a methanol feed is injected in stages into the alkylation catalyst at one or more locations downstream from the location of injection of the toluene.
